(19) United States
(12) Patent Application Publication
(10) Pub. No.: US 2015/0217056 A1
(43) Pub. Date: Aug. 6, 2015

(54) THERAPY SYSTEMS AND METHODS UTILIZING TISSUE OXYGENATION DETECTION

(71) Applicant: Stratos Group LLC, Seattle, WA (US)

(72) Inventors: Thomas Daniel Kadavy, Bellevue, WA (US); Allan H. Stephan, Seattle, WA (US)

(21) Appl. No.: 14/584,767

(22) Filed: Dec. 29, 2014

Related U.S. Application Data
(60) Provisional application No. 61/922,589, filed on Dec. 31, 2013.

(51) Int. Cl. A61M 5/172; A61H 31/00; A61M 16/00; A61B 5/1455; A61B 5/145

(52) U.S. Cl. CPC A61M 5/1723; A61B 5/14551; A61B 5/14546; A61M 16/0003; A61H 31/004; A61M 2205/3303

(57) ABSTRACT

Systems for controlling or aiding patient therapy are provided. The systems may include a tissue oxygenation measurement device and a therapy delivery apparatus that is controllable to assist in attaining a target tissue oxygenation level or range, whereby patient health is improved by optimizing delivery of therapy based on measured tissue oxygenation. The therapy delivery apparatus may include, for example, a drug delivery apparatus, a ventilating apparatus, a fluid delivery apparatus and/or a chest compression delivery apparatus. The tissue oxygenation measurement device may include a probe for determining the oxygenation of tissue, for example, muscle tissue, by optically interrogating the tissue. Related methods for guiding patient therapy and exercise training are also provided.

### BACKGROUND

1. Technical Field

The present disclosure relates to systems and methods utilizing in vivo tissue oxygenation detection to guide therapy or other activities.

2. Description of the Related Art

The benefits of noninvasive systems for detecting and/or monitoring for the presence of a particular analyte are well known in the art, and in particular in the biological arts. For example, in pulse oximetry a sensor is placed on a patient's body, e.g., the patient's finger. The sensor emits a red light and an infrared (IR) light sequentially through the patient, and detects the resulting light transmitted through the patient. The changing absorbance of each of the two wavelengths as the heart beats is measured and used to determine the oxygenation of the pulsing arterial blood alone. The absorbance of the red and IR light is used to calculate the oxygenation of the blood. The level of detected oxygenation of the blood can in turn be used to guide patient therapy.

Known systems for detecting oxygenation and guiding therapy based upon the same, however, suffer from a variety of deficiencies and drawbacks. For example, pulse oximetry may not be a viable option during bypass surgery or other situations when pulsatile flow to the body may be limited. Accordingly, there remains a need for improved systems, devices and methods for detecting in vivo tissue oxygenation of tissue and guiding therapy or other activities based upon the same.

### BRIEF SUMMARY

Embodiments described herein provide systems and methods which utilize in vivo tissue oxygenation detection to guide therapy or other activities. In some instances, the systems and methods may be used to modify a patient's tissue oxygenation toward a target level or range.

Example systems include a therapy delivery machine or other therapy delivery apparatus that is controllable to assist in attaining a target tissue oxygenation level or range, whereby patient health is improved by optimizing delivery of therapy based on measured tissue oxygenation. Example therapy delivery machines and apparatuses may include, for example, a drug delivery machine or apparatus, a ventilating machine or apparatus, a fluid delivery machine or apparatus and/or a chest compression delivery machine or apparatus, whereby drugs, oxygen, intravenous fluids and/or chest compressions may be delivered based at least in part on measured tissue oxygenation of a patient or other targeted user.

Example systems may further include a tissue oxygenation measurement device, such as an oxygenation measurement probe, for determining the oxygenation of tissue (e.g., muscle tissue) by optically interrogating the tissue. The tissue oxygenation measurement device may be configured to noninvasively assist in determining the oxygenation of the tissue by optically interrogating the tissue in both a visible wavelength range and a near infrared (NIR) wavelength range. The illuminating light may be sculpted in intensity to approximately match the absorbance spectrum, for example, with the visible light having an intensity of about an order of magnitude greater than the NIR light.

Example systems may further include a controller that is configured to measure an oxygenation level of the patient's tissue using the tissue oxygenation measurement device and the following ratio (hereinafter “Mox ratio”), which represents a weighted average of myoglobin and hemoglobin oxygen saturations:

oxymyoglobin+oxyhemoglobin(deoxymyoglobin+deoxyhemoglobin)+(oxymyoglobin+oxyhemoglobin)

Related methods for guiding patient therapy are also provided. For example, according to one embodiment, a method for guiding patient therapy may be summarized as including measuring an oxygenation level of a patient's tissue using the Mox ratio and adjusting delivery of a pharmaceutical drug to the patient via a drug delivery apparatus, whereby the patient's tissue oxygenation is modified toward a target level. According to another embodiment, a method for guiding patient therapy may be summarized as including measuring an oxygenation level of a patient's tissue using the Mox ratio and adjusting delivery of oxygen to the patient via a ventilating apparatus, whereby the patient's tissue oxygenation is modified toward a target level. According to another embodiment, a method for guiding patient therapy may be summarized as including measuring an oxygenation level of a patient's tissue using the Mox ratio and adjusting delivery of an intravenous fluid to the patient using a fluid delivery apparatus, whereby the patient's tissue oxygenation is modified toward a target level. According to yet another embodiment, a method for guiding patient therapy may be summarized as including measuring an oxygenation level of a patient's tissue using the Mox ratio and adjusting delivery of chest compressions to the patient, whereby the patient's tissue oxygenation is modified toward a target level. Other methods may include a method to monitor change in tissue oxygenation as a result of immune system response to an infectious microbe, a method to monitor change in tissue oxygenation as a result of circulatory system response to physical or emotional stress, and a method for guiding human exercise training or guiding recovery following exercise training.

### BRIEF DESCRIPTION OF THE SEVERAL VIEWS OF THE DRAWINGS

FIG. 1 is a perspective view of a system for guiding patient therapy, according to one embodiment.

FIG. 2 is a diagram of the system for guiding patient therapy of FIG. 1 interfaced with a patient.

FIG. 3 is a diagram of the absorbance spectrum of solutions of oxyhemoglobin and deoxyhemoglobin.

FIGS. 4 and 5 show opposing isometric views of an example tissue oxygenation measurement device in the form of a noninvasive probe, which may be used in connection with the therapy system of FIGS. 1 and 2.

FIG. 6 is an exploded view of the tissue oxygenation measurement device of FIGS. 4 and 5.

FIG. 7 is a perspective view of a system for guiding patient therapy, according to another embodiment, which includes a fluid delivery apparatus and/or drug delivery apparatus.

FIG. 8 is a perspective view of a system for guiding patient therapy, according to another embodiment, which includes a ventilating apparatus.

FIG. 9 is a perspective view of a system for guiding patient therapy, according to yet another embodiment, which includes a chest compression delivery apparatus.

FIG. 10 is a perspective view of a system for guiding human exercise training, according to one embodiment, which includes an exercise apparatus in the form of a treadmill.

### DETAILED DESCRIPTION

In the following description, certain specific details are set forth in order to provide a thorough understanding of various disclosed embodiments. However, one of ordinary skill in the relevant art will recognize that embodiments may be practiced without one or more of these specific details, or with other methods, components, materials, etc. In other instances, well-known structures, devices and features associated with therapy delivering systems and related therapy delivery methods have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense that is as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, aspects, structures, or characteristics described herein may be combined in any suitable manner in one or more embodiments.

Noninvasive methods, apparatuses, and systems for detecting an analyte in a medium such as a biological tissue and for guiding therapy in view of the same are disclosed. There are many biological and medical applications wherein a noninvasive means for determining oxygenation in tissue would provide benefits. For example, tissue oxygenation provides an early signal of potential problems in physiology and pathology. The systems and methods for monitoring muscle oxygenation and guiding therapy described herein can also be applied in clinical areas such as cardiac surgery, in sports medicine, and the like. In some embodiments, systems and methods for monitoring muscle oxygenation to provide early detection of shock and for guiding therapy to treat the same are provided.

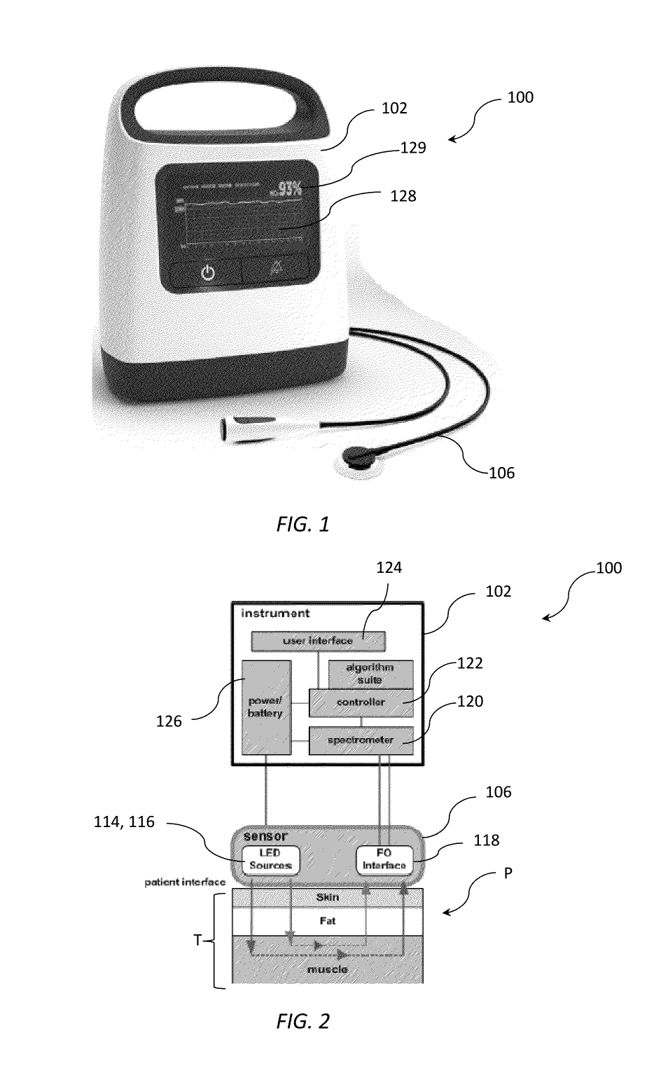

FIG. 1 shows an example system 100 for guiding therapy or other activities, according to one embodiment, which includes a therapy instrument 102 and a tissue oxygenation measurement device 106 coupled thereto for obtaining tissue oxygenation measurement information. The tissue oxygenation measurement device 106 may be in the form of a noninvasive probe communicatively coupled to the therapy instrument 102.

In some instances, the tissue oxygenation measurement device 106 may be configured to measure continuous wavelengths of light in both the visible spectrum and the near infrared (NIR) spectrum. The addition of at least a portion of the visible region of the spectrum allows enhanced sensitivity to muscle oxygenation and more rapid detection of circulatory shock, as described in more detail in U.S. patent application Ser. No. 13/883,279, which is incorporated herein by reference in its entirety. Visible light, as used herein, is expressly defined to comprise electromagnetic radiation having a wavelength in the range of 400-700 nm. Near infrared light, as used herein, is expressly defined to comprise electromagnetic radiation with a wavelength in the range of 700-3,000 nm.

FIG. 2 is a diagram providing an overview of the system 100 for guiding therapy or other activities, which includes measuring and/or monitoring the oxygenation of tissue in vivo and guiding patient therapy accordingly. As described above, the system 100 includes a therapy instrument 102 and a tissue oxygenation measurement device 106 for obtaining tissue oxygenation measurement information. For this purpose, the tissue oxygenation measurement device 106 may include a visible light source 114, an NIR light source 116, and a light detector 118. The visible light source 114 and NIR light source 116 may illuminate a desired region of tissue T of a patient P adjacent the tissue oxygenation measurement device 106. For example, visible and NIR light may transmit through a user's skin and fluids to illuminate the desired region of tissue T, as illustrated in FIG. 2.

The light detector 118 may receive light reflected from or transmitted through the desired region of tissue T, and transmit the detected light to a spectrometer, spectrophotometer, or the like 120, contained in the therapy instrument 102. Various types of spectrometers 120 are contemplated, including those having a sensor and a prism, diffraction grating, or slit. Both the prism (or grating or slit) and the sensor may be stationary or swept. For example, the system 100 may include a fiber-optic spectrophotometer 120 having a grating and a photodetector, such as a photodiode array, a charge-coupled device (CCD), or a complementary metal-oxide-semiconductor device such as an active pixel sensor (CMOS APS).

The therapy instrument 102 may further include a controller 122 for processing the detected spectra and storing data related to the same. The controller 122 may generally include, without limitation, one or more computing devices, such as processors, microprocessors, digital signal processors (DSP), application-specific integrated circuits (ASIC), and the like. To store information, the controller 122 may also include one or more storage devices, such as volatile memory, non-volatile memory, read-only memory (ROM), random access memory (RAM), and the like. The storage devices can be coupled to the computing devices by one or more buses. The controller 122 may further include one or more input devices (e.g., displays, keyboards, touchpads, controller modules, or any other peripheral devices for user input) and output devices (e.g., displays screens 128, as shown in FIG. 1, light indicators, and the like), collectively referred to as user interface 124. The controller 122 can store one or more programs for processing the detected spectra and an associated algorithm suite. The controller 122 may control operation of other components, such as, for example, the visible and NIR light sources 114, 116 described herein.

The controller 122, according to one embodiment, may be provided in the form of a general purpose computer system. The computer system may include components such as a CPU, various I/O components, storage, and memory. The I/O components may include a display, a network connection, a computer-readable media drive, and other I/O devices (a keyboard, a mouse, speakers, etc.). A control system manager program may be executing in memory, such as under control of the CPU, and may include functionality related to gathering and processing tissue oxygenation data. The therapy instrument 102 may further include a power source 126 for powering components thereof.

As described above, the controller 122 may control the visible and NIR light sources 114, 116. In one embodiment, for example, the visible light source 114 and the NIR light source 116 may be controlled to synchronously emit light intermittently. In other instances, the visible light source 114 and the NIR light source 116 may be controlled to emit light intermittently in an asynchronous manner. For example, the visible light source 114 may emit light intermittently during a period in which the NIR light source 116 is inactive and the NIR light source 116 may emit light intermittently during a different period in which the visible light source 114. The periods may be the same or different durations.

The controller 122 may include a processor configured to execute instructions for implementing an algorithm to process tissue oxygenation data obtained by the tissue oxygenation measurement device 106. The instructions, algorithm, and data can be stored in memory or received from an input. Again, the memory, in various examples, may include volatile or non-volatile memory or other storage devices. Typically, as shown in FIG. 1, a display device 128 may be provided to display the status of the therapy system 100 and/or the results of the analysis of the spectra, such as, for example, an indication of tissue oxygenation level 129 in a human understandable format. The display device 128 may also display a graph of tissue oxygenation levels over time. The therapy instrument 102 may further include a visual or audible alert to provide a notification if the results of the analysis indicate the need for remedial action. In some instances, as described in more detail elsewhere, the therapy instrument 102 may be coupled to or integrated with one or more therapy delivery devices (e.g., drug delivery apparatus, ventilating apparatus) to deliver a desired type of therapy (e.g., pharmaceutical drugs, oxygen) in response to measured tissue oxygenation levels.

In an embodiment adapted to monitor for shock, the tissue oxygenation measurement device 106 may be attachable to the patient, for example, over the thenar muscle group in the hand. The visible and NIR light sources 114, 116 may illuminate muscle tissue, and the reflected light returning to the spectrometer 120 may be recorded. The spectra may be provided to the controller 122 for analysis, as described below, to calculate muscle oxygenation (Mox). If the Mox indicates that the patient is experiencing shock, a caregiver may be alerted to take appropriate action. In other instances, the therapy instrument 102 may be coupled to or integrated with one or more therapy delivery devices (e.g., drug delivery apparatus, ventilating apparatus) to deliver a desired type of therapy (e.g., pharmaceutical drugs, oxygen) in response to measured tissue oxygenation.

Mox has been found to be an early indicator of shock. Moreover, Mox can be used to differentiate between mild, moderate, and severe shock. Low Mox in critically ill patients can be used to identify the early stages of shock since blood flow is preferentially shunted away from skin and muscle as the body tries to preserve blood flow to the critical organs of the body—the heart, brain, and liver.

Mox is a measure of cellular oxygenation in muscle tissue and is a weighted average of myoglobin and hemoglobin oxygen saturations. The weighting depends on the relative concentrations of total myoglobin and total hemoglobin in the light path.

In some embodiments, the controller 122 may be configured to perform analyses to calculate tissue oxygenation defined as myoglobin saturation. Since myoglobin is contained within muscle cells, myoglobin saturation is a measure of intracellular oxygenation that will be useful in the detection of shock and the monitoring of critically ill patients.

In other embodiments, tissue oxygenation can be defined as hemoglobin saturation. Hemoglobin saturation measurements would not depend on the patient having pulsatile blood flow; it is an aggregate saturation measurement of pulsatile arterial blood, non-pulsatile arterial blood, and venous blood. Hemoglobin saturation can be measured in muscle tissue and in other tissues that do not contain myoglobin, such as the brain.

In some embodiments of the system 100 for guiding therapy or other activities, the tissue oxygenation measurement device 106 may be configured to illuminate the tissue T with a sculpted or shaped intensity (or radiant flux) illumination that improves the usefulness of the reflectance or transmission of the optical spectral information obtained. For example, in one embodiment the visible light source 114 is configured to illuminate the desired tissue with light in a visible region having a radiant flux that is an order of magnitude larger than the NIR illumination provided by the NIR light source 116. By matching the profile of the incident illumination to the expected absorbance of the tissue, the signal-to-noise ratio of the detected spectra can be optimized, resulting in higher quality optical spectra than can be acquired with a traditional broadband light source. It is contemplated that the visible light source 114 will produce a significantly larger radiant flux than the NIR light source 116, although the difference may be less than, or greater than, an order of magnitude.

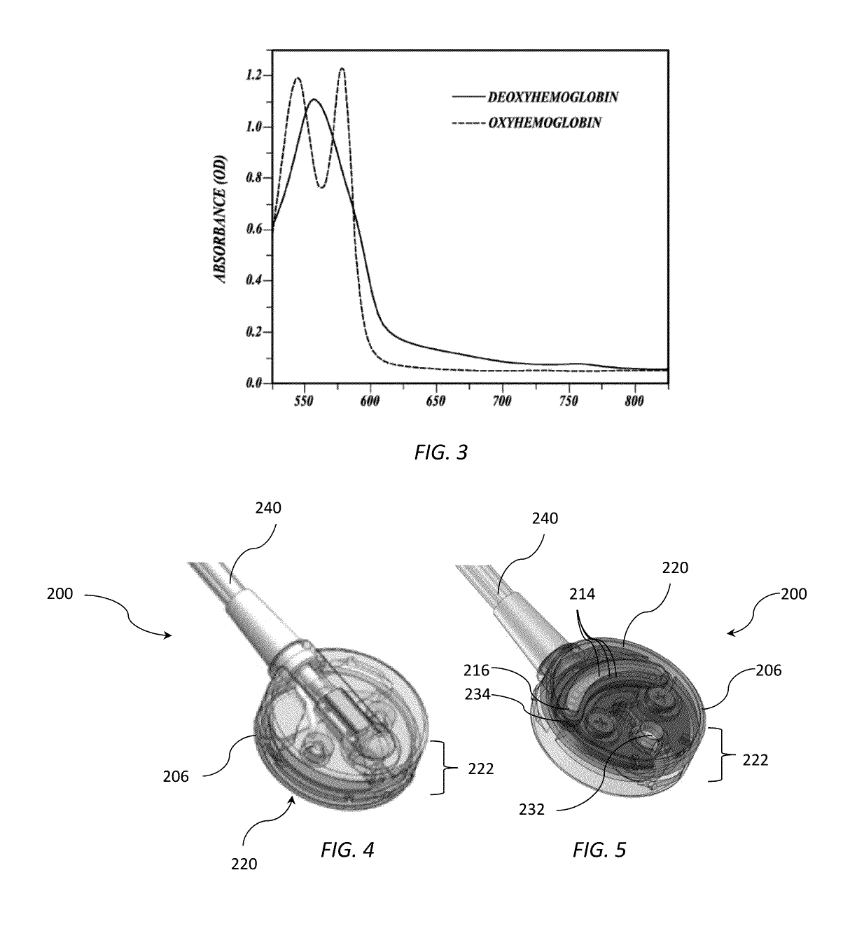

To understand the reason for the improved performance from the sculpted illumination, reference is made to FIG. 3, which shows the absorbance spectrum of solutions of oxyhemoglobin and deoxyhemoglobin. Hemoglobin in the blood binds with oxygen and carries oxygen from the lungs to the body tissues where it releases the oxygen and carries the resultant carbon dioxide back to the lungs. Oxyhemoglobin is formed when hemoglobin binds with oxygen; deoxyhemoglobin is formed when no oxygen molecules are bound to hemoglobin. Myoglobin is an oxygen-binding protein in muscle tissue and may similarly be bound to oxygen molecules (oxymyoglobin) or not bound to oxygen molecules (deoxymyoglobin).

It can be seen from FIG. 3 that the absorbance spectrum for oxyhemoglobin differs from the absorbance spectrum for deoxyhemoglobin. The differences in the absorbance characteristics may be used to optically evaluate the oxygenation of the hemoglobin. The ability to accurately evaluate the oxygenation can be improved by looking at features of the oxygenation curve in both the visible light region and the NIR region. However, the peaks of the absorbance or optical density for oxyhemoglobin in the visible region (and more particularly at about 545 nm about 580 nm) are about an order of magnitude larger than the peak of the absorbance of deoxyhemoglobin in the NIR region (e.g., at approximately 760 nm). Therefore, if a conventional broadband light source is used, the amount of light collected in the visible region will be about ten times smaller than that collected in the NIR region. Moreover, FIG. 3 illustrates a best-case scenario in which absorbance spectra are collected from a solution of hemoglobin that lacks a scatterer. When light scattering is significant, as in biologic tissue, the collected light from the visible region may be as much as three orders of magnitude smaller than in the NIR region. The absorbance spectrum for oxymyoglobin and deoxymyoglobin also differ in a qualitatively similar way. The differences in the absorbance spectra can be analyzed to calculate tissue oxygenation (e.g., Mox, myoglobin saturation, or hemoglobin saturation).

Accordingly, in some embodiments, the light sources 114, 116 of the tissue oxygenation measurement device 106 may be configured to produce a sculpted illumination having an intensity or radiant flux profile that is generally matched to the expected absorbance spectrum. In other words, the radiant flux of the illumination in wavelength regions with high absorbances may be higher than the radiant flux of the illumination in wavelength regions with lower absorbances. In some embodiments, for example, the visible light source 114 may be configured to illuminate the target tissue T with a radiant flux that is an order of magnitude, or more, greater than the corresponding radiant flux produced by the NIR light source 116. In other instances, other illumination and detection patterns and techniques may be utilized.

A number of different methods may be used to approximately match the illuminating light with the absorbance characteristics of the target. For example, filters may be used with a broadband light source to produce the desired sculpted illumination.

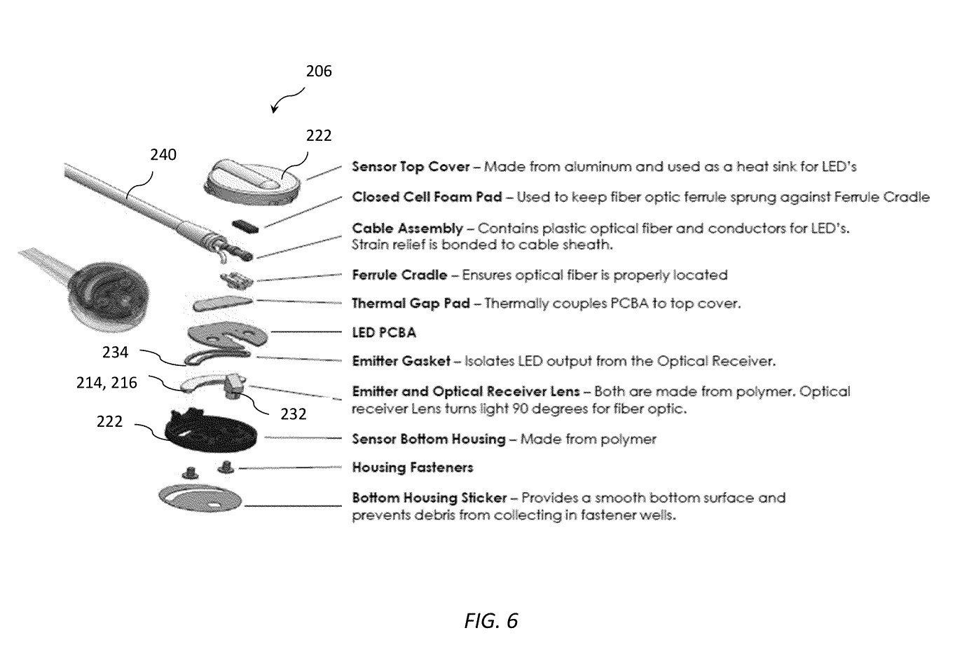

With reference to FIGS. 4 through 6, and according to one example embodiment, a tissue oxygenation measurement device 200 in the form of a noninvasive probe 206 may be provided for use in connection with the systems and methods disclosed herein. The probe 206 may include a visible light source 214 and a NIR source 216. The visible light source 214 and the NIR light source 216 may be implemented with light-emitting diodes (LED) disposed to emit light from a distal face 220 of the probe 206. Although the disclosed probe 206 uses LEDs that are disposed to emit light from a distal face 220 of the probe 206, other configurations are contemplated. For example, in other embodiments, light sources may be disposed away from the probe 206 and light may be transmitted to the probe 206 through fiber optic cables or the like.

With continued reference to FIGS. 4 through 6, the probe 206 may include a housing 222 that is configured to be placed against the user or tissue T to be examined. The housing 222 may be rigidly constructed or may be conformable to the user. The visible light source 214 may comprise a plurality of LEDs that emit light in visible wavelengths (e.g., 540-620 nm), and the NIR light source 216 may comprise a single LED that emits in the NIR wavelengths (e.g., 740-790 nm). The probe 206 may be configured to illuminate the target tissue T such that the intensity of the visible light illumination is approximately an order of magnitude greater than the intensity of the NIR illumination.

A light detector 232, such as, for example, a conventional CMOS photodetector, may be positioned in the housing 222 to detect light reflected from the target tissue T. Advantageously, the visible light LEDs 214 and the NIR LED 216 may all be disposed along a circular arc centered on the detector 232 such that the LEDs 214, 216 are all the same distance from the detector 232. The distance from the LEDs 214, 216 to the detector 232 may be selected to optimize light collection, depending on characteristics of the tissue T and the spectral information being collected. In particular, the distance between the LEDs 214, 216 and the detector 232 may be selected to provide a desired depth of penetration into the tissue T.

Although it is not critical, it should be appreciated that the LEDs 214, 216 in the example embodiment shown in FIGS. 4 through 6 are disposed along a circular arc that comprises approximately a 90° arc segment rather than a complete circle centered on the detector 232. This configuration advantageously allows for relatively large spacing between the LEDs 214, 216 and the detector 232 (e.g., 15-20 mm), while keeping the size of the probe 206 reasonable. The LEDs 214, 216 and/or the detector 232 are preferably disposed in the housing 222 such that the detector 232 is shielded from stray light. For this purpose, a shield or gasket 234 may be provided around the LEDs 214, 216 to isolate the detector 232 from stray light.

The probe 206 may further include a cable assembly 240 for communicatively coupling the probe 206 to the spectrometer 120 (FIG. 2) to enable the gathering and processing of data indicative of tissue oxygenation. The cable assembly 240 may also be communicatively coupled to the controller 122 (FIG. 2) for controlling the operation of the visible and NIR LEDs 214, 216.

In other instances, visible and NIR light sources may be provided remotely from the face 220 of the probe 206, and a first fiber optic system may be used to transmit light to the face 220 of the probe 206, and a second fiber optic system may return light from the detector 232 to the spectrometer 120 (FIG. 2).

Advantageously, the probe 206 may be configured to collect reflectance spectral information in a noninvasive manner. The probe may be used to collect, for example, reflectance optical spectra in humans or other mammals for medical diagnostics, exercise physiology, or a wide variety of other applications.

Again, it has been found that Mox measurement is effective for the early detection of shock, including septic shock. As an example, spectra may be acquired from the thenar muscles of healthy human subjects to build a locally weighted regression (LWR) model that may be used to provide real-time measurement of Mox in other subjects. LWR is a nonparametric learning algorithm that modifies a conventional linear or nonlinear least squares regression model by introducing a weighting scheme to give greater effect to “local” data points, and less weight to more distant data points.

In some embodiments, the systems and methods described herein may involve the analysis of concatenated portions of the spectrum data around known peaks for oxymyoglobin, deoxymyoglobin, oxyhemoglobin, and deoxyhemoglobin, which can enhance the analysis using a spectral methods approach, such as Multivariate Curve Resolution (MCR). Measurement of reflected light in the visible region around 580 nm yields information about concentrations of oxymyoglobin and oxyhemoglobin because these chromophores absorb in this region. Absolute quantification of myoglobin and hemoglobin saturation is made possible by the addition of measurements of concentrations of deoxymyoglobin and deoxyhemoglobin. These chromophores absorb in the NIR region around 760 nm.

The NIR portion of each concatenated spectrum may be scaled up relative to the visible portion of the spectrum. The magnitudes of the NIR absorbances are much smaller than those of the visible absorbances. Although the scaling step is not critical, without scaling the normalization may bias MCR toward erroneously oxygenated Mox values.

MCR may be used to obtain Mox values for each spectrum in a data set. MCR is an iterative spectral analysis method that can determine the individual concentrations of absorbing species within complex spectra. In some embodiments, in each iterative step, MCR simultaneously quantifies concentrations of two components in each spectrum, oxyMbHb and deoxyMbHb, where by definition:

[oxyMbHb]=[oxymyoglobin]+[oxyhemoglobin],

and

[deoxyMbHb]=[deoxymyoglobin]+[deoxyhemoglobin].

In every iterative step, MCR also uses the current estimates of component concentrations to determine the shape of the pure component spectra. When MCR has converged on the shape of the pure component spectra of oxyMbHb and deoxyMbHb and their relative concentrations in each spectrum in the data set, Mox is calculated from each spectrum in a subject data set as:

Mox={[oxyMbHb]/([oxyMbHb]+[deoxyMbHb])}*100

Myoglobin saturation (Mb sat) may also be calculated from MCR determination of [oxyMb] and [deoxyMb]:

Mb sat={[oxyMb]/([oxyMb]+[deoxyMb])}*100

The concentrations of [oxyHb] and [deoxyHb] can also be determined by MCR, resulting in a calculation of hemoglobin saturation (Hb sat):

Hb sat={[oxyHb]/([oxyHb]+[deoxyHb])}*100

The concentrations [oxyMb], [deoxyMb], [oxyHb], and [deoxyHb] may be determined simultaneously using MCR. Hemoglobin saturation may be determined using MCR in tissues where myoglobin is not present.

Spectra from a number of subjects and associated Mox, Mb sat, or Hb sat values for each spectrum, determined with MCR, may form a training set. The set of training data calculated from the test subjects may then be used to build an LWR model, which can provide for real-time monitoring or testing of patients or other subjects. When spectra are collected from a patient, the spectra may be preprocessed in the same way the LWR training set spectra were processed, including taking the second derivative, concatenating selected portions, scaling the NIR portion, normalization, and mean centering.

A preferred method uses locally weighted regression (LWR) with partial least squares (PLS) techniques to calculate Mox from spectra in real time. In particular, for each new spectrum acquired from a patient, LWR builds a local PLS model from spectra in the in vivo training set that are most similar to the new spectrum. The LWR method does not allow, for example, training set spectra with vastly different melanin and lipid characteristics from the new spectrum to contribute to its Mox measurement. This essentially creates an effective filter for spectrum characteristics that reflect melanin and lipid content and that are unrelated to Mox. Mox may be calculated from each test set spectrum by application of the local PLS model.

When spectra in the LWR training set encompass a wide range of physical characteristics found in patients, the robustness of the Mox measurement is improved by assuring that appropriate training set spectra are available. Good accuracy in human subjects has been demonstrated using LWR with a relatively small training set. Other types of LWR models can be built to measure tissue oxygenation. Mb sat may be measured from spectra in the training set and used to build an LWR model that will measure Mb sat from patient spectra. Similarly, Hb sat may be measured from training set spectra collected from tissue that either does or does not contain myoglobin. These Hb sat values may be used with the training set spectra to build an LWR model that will measure Hb sat from patient spectra.

Obtaining and monitoring Mox measurements may prove beneficial in numerous settings, such as, for example, the detection of shock, and can be instrumental in the early and accurate detection of abnormal tissue oxygenation levels. In addition, tissue oxygenation monitoring may be beneficial in exercise physiology and sports medicine applications, such as, for example, providing an indicator of aerobic capacity, muscle anaerobic metabolism, and performance.

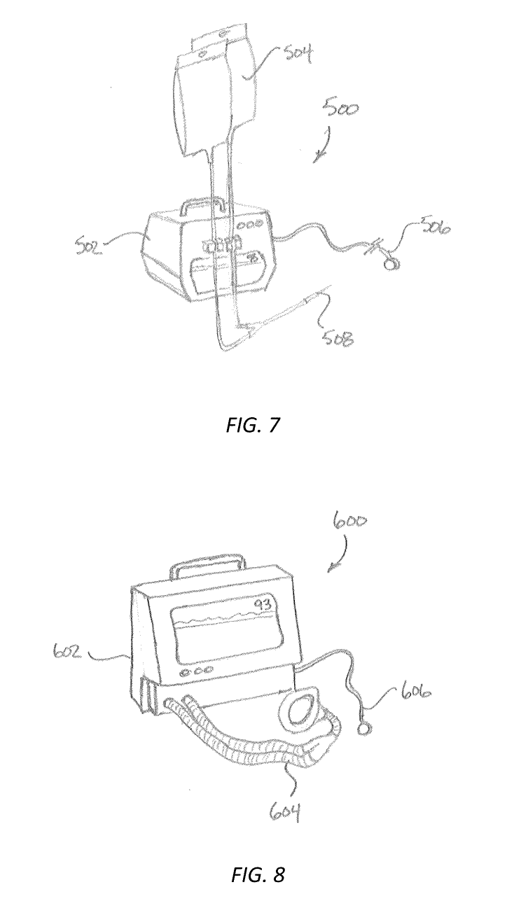

FIGS. 7 through 10 illustrate various systems (and related methods) that involve obtaining and monitoring Mox measurements for purposes of guiding therapy or other activities.

FIG. 7, for example, illustrates a system 500 for guiding patient therapy, which includes a noninvasive probe 506 for detecting in vivo tissue oxygenation in a patient and a fluid delivery and/or drug delivery apparatus 502 that may be adjusted manually or automatically based at least in part on tissue oxygenation measurements. The fluid delivery and/or drug delivery apparatus 502 may include a fluid and/or drug source 504 that may be supplied intravenously to a patient (not shown) via a catheter assembly 508. The fluid delivery and/or drug delivery apparatus 502 may further include a controller that is communicatively coupled to the probe 506 and configured to measure an oxygenation level of the patient's tissue using the probe 506 and the following Mox ratio:

$$\frac{oxymyoglobin + oxyhemoglobin}{\begin{matrix}( deoxymyoglobin + deoxyhemoglobin ) + \\ ( oxymyoglobin + oxyhemoglobin )\end{matrix}} ,$$

which is a function of the oxygen status of both myoglobin and hemoglobin proteins in the target tissue. In some embodiments, the controller may be configured to automatically adjust the delivery of pharmaceutical drugs and/or intravenous fluids to a patient using the fluid delivery and/or drug delivery apparatus 502 to assist in modifying the patient's tissue oxygenation toward a target level or range, such as, for example, a Mox percentage of 94.0%±5.6.

A related method for guiding patient therapy may therefore include measuring an oxygenation level of a patient's tissue using the Mox ratio and adjusting delivery of a pharmaceutical drug and/or intravenous fluids to the patient, whereby the patient's tissue oxygenation is modified toward a target level or range. In some embodiments, adjusting delivery of the pharmaceutical drug and/or intravenous fluid to the patient via may include delivering a dosage of the pharmaceutical drug and/or the intravenous fluids in response to a decrease in the measured Mox ratio beyond a first threshold value. The first threshold value may be for example, fifty percent, sixty percent or seventy percent of a target level or an average level of a healthy individual. For example, when utilizing 94.0% as a target Mox percentage level, the first threshold may be 47.0%, 56.4% or 65.8% in some embodiments. The method may further include delivering a supplemental dosage of the pharmaceutical drug or additional intravenous fluids in response to a subsequent decrease in the measured ratio beyond the first threshold value. This may follow a period of partial recovery of muscle oxygenation toward the target level and a subsequent decline of the same. In some instances, the method may include delivering the pharmaceutical drug and/or intravenous fluids based at least in part on a trend of the measured Mox ratio over time. For example, periods characterized by a general decline of the Mox ratio may be followed by the introduction of the pharmaceutical drug and/or intravenous fluids, whereas periods characterized by a general recovering may result in the cessation of such resuscitation efforts. In some embodiments, the methods for guiding patent therapy may take into account a rate at which the Mox ratio changes over time. For example, periods characterized by rapid or steady decline of the Mox ratio may be followed by the introduction of the pharmaceutical drug and/or intravenous fluids, whereas periods characterized by rapid or steady recovering may result in the cessation of such resuscitation efforts.

In some instances, the method may include delivering the pharmaceutical drug and/or intravenous fluids during an interval in which traditional monitoring techniques, such as, for example, pulse oximetry, blood pressure monitoring and arterial lactate monitoring, are ineffective in identifying a need for therapy measures. For example, the method may include delivering the pharmaceutical drug and/or intravenous fluids during early stages of shock when arterial lactate levels and/or blood pressure levels are within a normal range or at a slightly elevated level. In other instances, the method may include delivering the pharmaceutical drug and/or intravenous fluids during a portion of a resuscitation period in which pulse oximetry measurements are generally insensitive to changes in muscle oxidation levels.

FIG. 8 illustrates another example system 600 for guiding patient therapy. The system 600 includes a noninvasive probe 606 for detecting in vivo tissue oxygenation in a patient and a ventilating apparatus 602 that may be adjusted manually or automatically to deliver oxygen based at least in part on said tissue oxygenation measurements. The ventilating apparatus 602 may include or be coupled to an oxygen source (not shown) for supplying oxygen to a patient (not shown) via an oxygen mask assembly 604. The ventilating apparatus 602 may further include a controller that is communicatively coupled to the probe 606 and configured to measure an oxygenation level of the patient's tissue using the probe 606 and the Mox ratio, which again is a function of the oxygen status of both myoglobin and hemoglobin proteins in the target tissue. In some embodiments, the controller may be configured to automatically adjust the delivery of oxygen to a patient using the ventilating apparatus 602 to assist in modifying the patient's tissue oxygenation toward a target level or range.

A related method for guiding patient therapy may therefore include measuring an oxygenation level of a patient's tissue using the Mox ratio and adjusting delivery of pure oxygen and/or air to the patient, whereby the patient's tissue oxygenation is modified toward a target level or range. In some embodiments, adjusting delivery of oxygen and/or air to the patient may include delivering oxygen and/or air in response to a decrease in the measured Mox ratio beyond a first threshold value. The first threshold value may be for example, fifty percent, sixty percent or seventy percent of a target level or an average level of a healthy individual. The method may further include delivering supplemental oxygen and/or air in response to a subsequent decrease in the measured ratio beyond the first threshold value. This may follow a period of partial recovery of the Mox ratio toward the target level and subsequent decline of the same. In some instances, the method may include delivering oxygen and/or air based at least in part on a trend of the measured Mox ratio over time. For example, periods characterized by a general decline of the Mox ratio may be followed by the introduction of supplemental oxygen or air, whereas periods characterized by a general recovering may result in the cessation of such resuscitation efforts. In some embodiments, the methods for guiding patent therapy may take into account a rate at which the Mox ratio changes over time. For example, periods characterized by rapid or steady decline of the Mox ratio may be followed by the introduction of supplemental oxygen or air, whereas periods characterized by rapid or steady recovering may result in the cessation of such resuscitation efforts.

In some instances, the method may include delivering oxygen and/or air during an interval in which traditional monitoring techniques, such as, for example, pulse oximetry, blood pressure monitoring and arterial lactate monitoring, are ineffective in identifying a need for therapy measures. For example, the method may include delivering oxygen and/or air during early stages of shock when arterial lactate levels and/or blood pressure levels are within a normal range or at a slightly elevated level. In other instances, the method may include delivering oxygen and/or air during a portion of a resuscitation period in which pulse oximetry measurements are generally insensitive to changes in muscle oxidation levels.

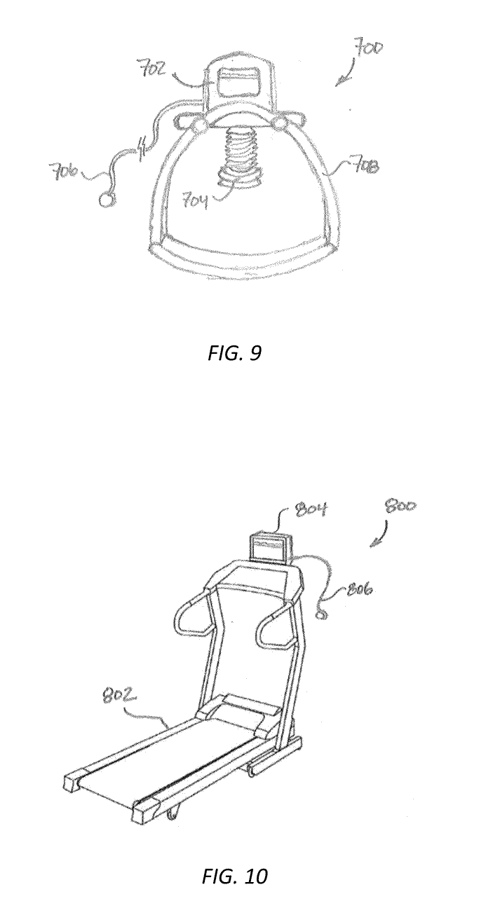

FIG. 9 illustrates yet another example system 700 for guiding patient therapy. The system 700 includes a noninvasive probe 706 for detecting in vivo tissue oxygenation in a patient and a chest compression delivery apparatus 702 that may be adjusted manually or automatically to deliver chest compressions to a patient based at least in part on said tissue oxygenation measurements. The chest compression delivery apparatus 702 may include a frame 708 and a chest compression delivery actuator 704 coupled thereto which is controllably movable to deliver chest compressions. The chest compression delivery apparatus 702 may further include a controller that is communicatively coupled to the probe 706 and configured to measure an oxygenation level of the patient's tissue using the probe 706 and the Mox ratio, which again is a function of the oxygen status of both myoglobin and hemoglobin proteins in the target tissue. In some embodiments, the controller may be configured to automatically adjust the delivery of chest compressions to a patient using the chest compression delivery apparatus 702 to assist in modifying the patient's tissue oxygenation toward a target level or range.

A related method for guiding patient therapy may therefore include measuring an oxygenation level of a patient's tissue using the Mox ratio and adjusting delivery of chest compressions to the patient, whereby the patient's tissue oxygenation is modified toward a target level or range. In some embodiments, adjusting delivery of chest compressions to the patient may include delivering chest compressions in response to a decrease in the measured Mox ratio beyond a first threshold value. The first threshold value may be for example, fifty percent, sixty percent or seventy percent of a target level or an average level of a healthy individual. The method may further include delivering supplemental chest compressions in response to a subsequent decrease in the measured ratio beyond the first threshold value. This may follow a period of partial recovery of the Mox ratio toward the target level and subsequent decline of the same. In some instances, the method may include delivering chest compressions based at least in part on a trend of the measured Mox ratio over time. For example, periods characterized by a general decline of the Mox ratio may be followed by the delivery of chest compressions, whereas periods characterized by a general recovering may result in the cessation of such resuscitation efforts. In some embodiments, the methods for guiding patent therapy may take into account a rate at which the Mox ratio changes over time. For example, periods characterized by rapid or steady decline of the Mox ratio may be followed by the delivery of chest compressions, whereas periods characterized by rapid or steady recovering may result in the cessation of such resuscitation efforts.

FIG. 10 illustrates an example system 800 for guiding exercise training. The system 800 includes a noninvasive probe 806 for detecting in vivo tissue oxygenation in a user and an exercise apparatus 802 in the form of a motorized treadmill that may be adjusted manually or automatically in terms of speed, inclination and/or resistance based at least in part on said tissue oxygenation measurements. The exercise apparatus 802 may include a control unit 804 having a controller that is communicatively coupled to the probe 806 and configured to measure an oxygenation level of the user's tissue using the probe 806 and the Mox ratio, which again is a function of the oxygen status of both myoglobin and hemoglobin proteins in the target tissue. In some embodiments, the controller may be configured to automatically adjust the speed, inclination and/or resistance of the treadmill to vary a training regimen provided in conjunction with the treadmill to assist in modifying the patient's tissue oxygenation either toward or away from a target level or range. For example, in some instances, the controller may be configured to automatically adjust the speed, inclination and/or resistance of the treadmill to assist in maintaining the user's tissue oxygenation level within a range that corresponds to that suitable for peak performance. In other instances, the controller may be configured to automatically adjust the speed, inclination and/or resistance of the treadmill to urge the user's tissue oxygenation level beyond a threshold level for endurance training purposes.

A related method for guiding exercise training may therefore include measuring an oxygenation level of a user's tissue using the Mox ratio and adjusting operational characteristics of the exercise apparatus, whereby the patient's tissue oxygenation is modified toward or away from a target level or range. In some embodiments, the methods for guiding exercise training may take into account a rate at which the Mox ratio changes over time in addition to changes in magnitude.

Still further, in other embodiments, the example system 800 for guiding exercise training may include a ventilating apparatus (not shown) that is configured to supply supplemental oxygen or air during and/or after a training session. In some instances, oxygen or air may be provided in response to measured Mox levels to speed recovery post exercise. In yet other embodiments, the probe 806 may be used in connection with a fitness assessment program to measure and provide an assessment of health or fitness in response to a pre-established fitness test training session.

These and other systems and related methods that involve obtaining and monitoring Mox measurements for purposes of guiding therapy and a wide range of other activities, such as guiding exercise training, are contemplated. For example, a method to monitor change in tissue oxygenation as a result of immune system response to an infectious microbe may be provided, which includes measuring an oxygenation level of the patient's tissue using the Mox ratio and presenting the ratio in a human understandable format, whereby a level of immune system response can be ascertained based at least in part on said ratio. Said ratio may be a numerical value and may be trended over time. Said ratio may also be compared to a prior established baseline value to ascertain a level of immune system response. Said immune system response may include systemic inflammation due to the presence of the infectious microbe which negatively influences the oxygenation level of the patient's tissue. In other instances, said immune system response may be due to the presence of sepsis or septic shock caused by the infectious microbe. As another example, a method to monitor change in tissue oxygenation as a result of circulatory system response to physical or emotional stress may be provided, which includes measuring an oxygenation level of the patient's tissue using the Mox ratio and presenting said ratio in a human understandable format, whereby a level of said physical or emotional stress can be ascertained. Said ratio may be a numerical value and may be trended over time. Said ratio may also be compared to a prior established baseline value to ascertain a level of circulatory system response to physical or emotional stress.

Still further, although some embodiments described herein are directed to therapy systems and methods including calculations of muscle oxygenation in particular, it will be readily apparent to persons of ordinary skill in the relevant art that the therapy systems and methods may be extended according to the teachings of the present disclosure to calculate the oxygenation of other tissues where there is no myoglobin, such as in the brain. In such applications rather than using the detected absorbance spectrum of muscle, the spectrum of a mixture of arterial, capillary, and venous hemoglobin could be analyzed generally using one or all of a suitably sculpted light sources, concatenated spectrum segments, spectral method to develop training data, and an LWR method to determine the oxygenation of the tissue that is not pulsatile arterial blood. Having obtained suitable training data, real time, specific and noninvasive tissue oxygenation measurements may be gathered for a variety of therapy and other purposes.

Moreover, the various aspects of the embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet, including U.S. patent application Ser. No. 13/883,279, are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for guiding patient therapy, the method comprising:
measuring an oxygenation level of a patient's tissue using the following ratio:
$$\frac{oxymyoglobin + oxyhemoglobin}{\begin{matrix}( deoxymyoglobin + deoxyhemoglobin ) + \\ ( oxymyoglobin + oxyhemoglobin )\end{matrix}} ;$$
and
adjusting delivery of a pharmaceutical drug to the patient via a drug delivery apparatus, whereby the patient's tissue oxygenation is modified toward a target level.

2. The method of claim 1 wherein adjusting delivery of the pharmaceutical drug to the patient via the drug delivery apparatus includes delivering a dosage of the pharmaceutical drug in response to a decrease in the measured ratio beyond a first threshold value.

3. The method of claim 2 wherein adjusting delivery of the pharmaceutical drug to the patient via the drug delivery apparatus includes delivering a supplemental dosage of the pharmaceutical drug in response to a subsequent decrease in the measured ratio beyond the first threshold value.

4. The method of claim 1 wherein adjusting delivery of the pharmaceutical drug to the patient via the drug delivery apparatus includes delivering the pharmaceutical drug based at least in part on a trend of the measured ratio over time.

5. A system for guiding patient therapy, the system comprising:
a probe for detecting in vivo tissue oxygenation in a patient;
a drug delivery apparatus; and
a controller coupled to the probe and the drug delivery apparatus, the controller configured to measure an oxygenation level of the patient's tissue using the probe and the following ratio:
oxymyoglobin+oxyhemoglobin
(deoxymyoglobin+deoxyhemoglobin)+(oxymyoglobin+oxyhemoglobin), and to adjust delivery of a pharmaceutical drug to the patient via the drug delivery apparatus based at least in part on said ratio, whereby the patient's tissue oxygenation is modified toward a target level.

6-21. (canceled)

22. A system for guiding therapy of a patient in shock, the system comprising:
a probe for detecting in vivo tissue oxygenation in a patient;
a fluid delivery apparatus;
a ventilating apparatus;
a drug delivery apparatus; and
a controller coupled to the probe, the fluid delivery apparatus, the ventilating apparatus and the drug delivery apparatus, the controller configured to measure an oxygenation level of the patient's tissue using the probe and the following ratio:
oxymyoglobin+oxyhemoglobin
(deoxymyoglobin+deoxyhemoglobin)+(oxymyoglobin+oxyhemoglobin), and to adjust delivery of intravenous fluid, oxygen and a pharmaceutical drug to the patient using the fluid delivery apparatus, the ventilating apparatus and the drug delivery apparatus, respectively, whereby the patient's tissue oxygenation is modified toward a target level.

23. A system for controlling or aiding patient therapy, the system comprising:
a tissue oxygenation measurement device; and
a therapy delivery apparatus that is controllable to assist in attaining a target tissue oxygenation level or range, whereby patient health is improved by optimizing delivery of therapy based on measured tissue oxygenation.

24. The system of claim 23 wherein the therapy delivery apparatus is configured to provide chest compressions for purposes of cardio pulmonary resuscitation.

25. The device of claim 23 wherein the therapy delivery apparatus provides pharmaceutical drugs.

26. The system of claim 23 wherein the therapy delivery apparatus provides intravenous fluids.

27. The system of claim 23 wherein the therapy delivery apparatus provides oxygen gas.

28. The system of claim 23 wherein the therapy delivery apparatus provides oxygen gas and air.

29-41. (canceled)
